(19) United States
(12) Patent Application Publication
(10) Pub. No.: US 2006/0084929 A1
(43) Pub. Date: Apr. 20, 2006

(54) Infusion port

(76) Inventor: Kenneth Eliasen, East Bridgewater, MA (US)

Correspondence Address: GROSSMAN, TUCKER, PERREAULT & PFLEGER, PLLC, 55 SOUTH COMMERICAL STREET, MANCHESTER, NH, 03101, US

(21) Appl. No.: 10/890,909

(22) Filed: Jul. 13, 2004

(51) Int. Cl. A61M 31/00; A61M 37/00

(52) U.S. Cl. 604/288.02; 604/288.04

(57) ABSTRACT

An access portal is provided including a housing, a body defining a fluid reservoir, and a septum enclosing the fluid reservoir. A stem may be provided in fluid communication with the fluid reservoir through an outlet passage extending form the fluid reservoir. The access portal may also include at least one volume reduction member disposed within the fluid reservoir. The volume reduction member within the fluid reservoir may reduce the fluid fill volume of the fluid reservoir.

### FIELD

The present disclosure is generally directed at subcutaneously implantable vascular access portals. More specifically, the present disclosure is directed at vascular access ports having a reduced internal fluid fill volume.

### BACKGROUND

Direct access to the vascular system is a quick and effective way to administer a variety of drug therapies, provide nutrition, and/or sample blood. Currently, regular access to the vascular system is gained by using a device specifically designed for this task. Several types or families of these devices exist in the market today. Among them are needles, catheters and a group of devices known as implanted access portals.

Vascular access has evolved through the years to improve treatment of a number of chronic and non-chronic diseases. Needles have been used for many years to inject vaccines and antibiotics or withdraw blood. Although still widely used today, needles have several limitations that do not allow them to be used with all therapies. In the early 1970's the use of vascular access catheters was perfected. Vascular access catheters allowed long term antibiotic, chemo, and nutritional therapies to be administered without having to change the access device. Additionally, vascular access catheters made it possible to introduce a medicament into a large enough vessel to allow the hemo-dilution required for some of the more toxic therapeutic drugs. This type of catheter provides a significant improvement over needles for long-term access, however, the external segment of such catheters may be prone to infection and requires constant maintenance. The latest development in vascular access is the implanted access portal, or ports. These portals eliminate the need for an external segment and therefore do not have the drawbacks of catheters.

Although considered new technology in the field of vascular access, implanted access portals have existed in the market for over 20 years. Use of these products has increased dramatically during this period because they are generally the device of choice for long-term vascular access. They are particularly suited for long-term use because the entire device is implanted under the skin. Implantability is one factor in the success of the implantable access portals because it allows the patient to perform ordinary daily task such as bathing and swimming without worrying about harming an external segment of an access device or increasing the chance of infection. Thus the quality of life for the patient is improved and the clinician is presented with fewer device related complications.

Typically implanted access portals consist of a housing, a self-sealing septum, and an attachable or pre-connected catheter. Portal housings can be made of a variety of materials including plastic, metal, or a combination of both. The self-sealing septum is generally made of an elastomer such as silicone. Catheters are also generally made of a highly flexible material such as silicone or polyurethane. Different materials are used to manufacture the components to achieve certain desired characteristics in the portal (i.e. plastic is not radiopaque, therefore the port will not show up on fluoroscopy).

Implanted access portals are also designed in such a way that their size (height and footprint), shape, and number of lumens are appropriate for the intended use. Number of lumens can be critical if a patient requires simultaneous infusion of incompatible solutions or isolation of blood sampling. As concurrent therapies become more popular the need for a wider variety of dual-lumen ports has increased.

During the life cycle of an implanted infusion portal a variety of complications can arise that may limit its functionality or render it useless altogether. Among these complications is “sludge buildup” or unwanted buildup of precipitate in the portal reservoir. This buildup is generally caused by improper or inadequate flushing of the portal. Typically portals are cleared or flushed immediately after aspiration or infusion. Routine flushing or maintenance is also performed when a portal is used infrequently or not at all. Flushing usually consists of injecting saline solution or saline solution containing an anti-coagulant, such as Heparin, through the reservoir and out the catheter into the vascular system. Generally the instructions for use will specify the volume of fluid and frequency required for proper maintenance.

### BRIEF DESCRIPTION OF DRAWINGS

Features and advantages of the present invention are set forth by the description of exemplary embodiments consistent therewith, which description should be understood in conjunction with accompanying drawings, wherein:

FIG. 1 is a perspective view of an exemplary embodiment of a vascular portal according to the present disclosure;

FIG. 2 is an exploded perspective view of the exemplary vascular portal of FIG. 1

FIG. 3 is a perspective view of a body portion of one exemplary embodiment of a vascular access portal according to the present disclosure;

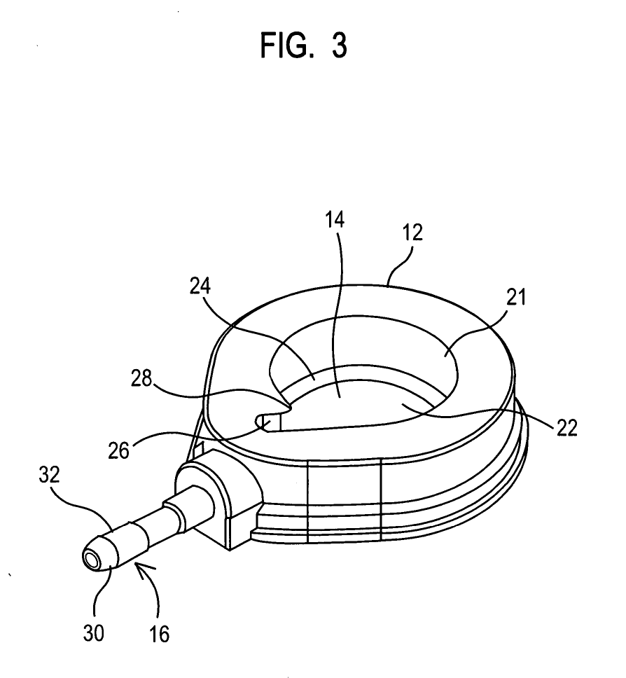

FIG. 4 is a plan view of a body portion of an exemplary access port shown in FIG. 3; and

FIG. 5 shows another embodiment of an access port in plan view.

### DESCRIPTION

The present disclosure is directed at an implantable vascular access portal, herein also referred to as a port. Particularly, according to one aspect this disclosure is directed at an access port that may reduce the accumulation of precipitate within the port. According to another aspect, the disclosed port may be designed to reduce turbulent flow of a fluid passing between the portal and an outlet.

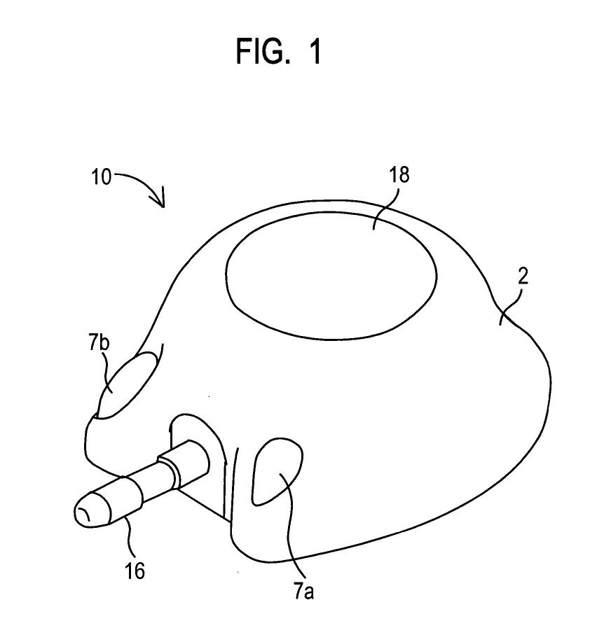

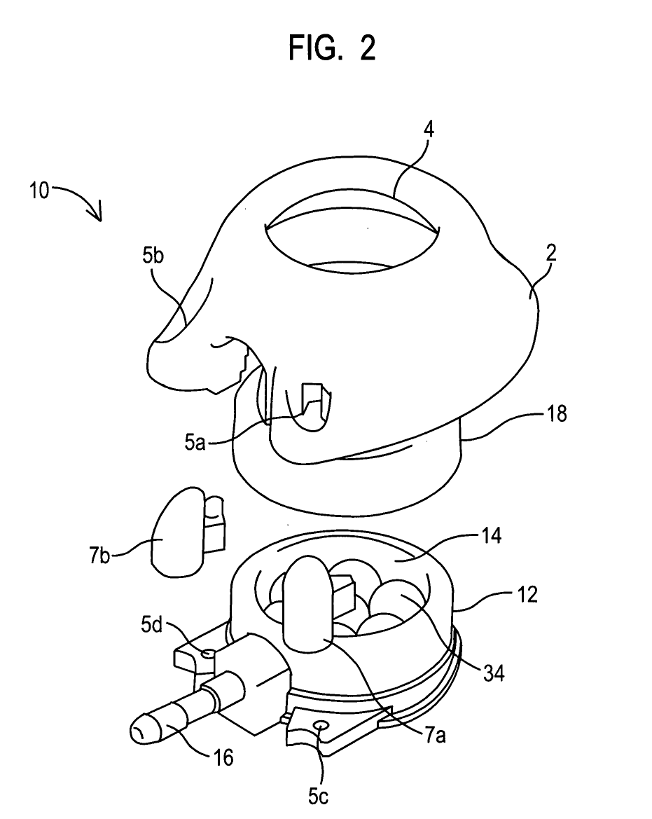

Turning to FIGS. 1 and 2, an exemplary access portal 10 consistent with the present disclosure is illustrated. As shown, the access portal 10 may generally include a housing 2, a body portion 12, a stem 16 and a septum 18. The housing 2 may define an opening 4 on a top surface thereof. The opening 4 in the housing 2 may permit access to a reservoir 14 defined in the body portion 12. The reservoir may be enclosed by the septum 18. The septum 18 may be disposed over the reservoir 14 between the body portion 12, and the housing 2.

The housing 2 may be constructed of any suitable biocompatible material, including polymeric materials, metallic materials, and ceramics. For example, the housing may conveniently be injection molded from a polymeric material and may generally define the overall profile and geometry of the access portal 10. The housing 2 may include a rounded or angled margin around the opening 4 to urge a needle downward toward the septum 18 and reservoir 14 covered thereby. This feature may reduce errant entry of needles within the septum 18.

The housing 2 may be assembly to the body 12 using a variety of techniques. As illustrated in FIG. 2, the housing 2 and the body 12 may include cooperating snap-fit features (not shown) or press-fit features. Accordingly, the housing 2 and body 12 may snap or press together. In addition, or as an alternative, to press or snap-fit assembly, the housing 2 and body 12 may be adhesively bonded together or welded, etc.

As shown, the access portal 10 may also include suture holes 5a-b extending through the housing 2 and corresponding suture holes 5c-d extending through the body 12. The suture holes 5a-d may allow the access portal 10 to be secured in a desired location within a patient. The access portal 10 may be secured by suturing through the suture holes 5a-d and tissue at the desired implantation site of the portal 10. Suture plugs 7a-b may be received in at least the suture holes 5a-b in the housing 2. The suture plugs may be employed to prevent the ingrowth and/or accumulation of tissue or other biometric material in the suture holes 5a-b of the housing 2. The suture plugs 7a-b may also be configured to be at least partially received in the suture holes 5c-d of the body 12. The suture plugs 7a-b may be formed of an elastomeric material, e.g., silicone, such that the suture plugs 7a-b may be penetrated with a suture needle and receive a suture passing through the plugs 7a-b. The elastomeric material may conform around the a suture passing therethrough

The body 12 may be formed from any suitable biocompatible material. Exemplary materials may include polymeric materials, stainless steel, titanium, ceramic, etc. The body 12 may also be formed from more than one material. For example, the body 12 may include a biocompatible polymer having a stainless steel, titanium, or ceramic insert defining at least a portion of the reservoir 14.

The stem 16 may provide an outlet from the reservoir 14, allowing fluids to be delivered to a predetermined location in the body. In a similar manner, the stem 16 may allow fluids to be delivered from a predetermined location in the body to the reservoir 14, e.g., to permit aspiration. Generally, delivery of fluids between the access port 10 and a predetermined location in the body may be accomplished, for example, by transporting the fluid through a catheter (not shown). It should be understood that a catheter may be implanted in the body of a patient extending between the portal site and the predetermined location in the body of the patient. Accordingly, the stem 16 may be configured to be received in a lumen of a catheter. The distal end of the stem 16 may include a tapered lead in 30. The distal end of the stem 16 may also include a bullet 32 or a barb for retaining a catheter to the stem 16.

The septum 18 generally encloses the reservoir 14, thereby retaining contents of the reservoir 14. Additionally, the septum 18 may permit the reservoir 14 to be accessed, e.g., transcutaneously using a hypodermic needle. Access to the reservoir 14 may permit the delivery of fluids to, or extraction of fluids from, the portal 10. Consistent with the function of providing access to the portal 10, the septum 18 may be formed from a needle penetrable material that is self sealing. Exemplary septum materials may include biocompatible elastomers, such as silicone, polyurethane, etc.

Consistent with one embodiment, the septum 18 may be compressed against the body 12 by the housing 2 sufficiently to seal the septum 18 to the body 12 around the perimeter of the reservoir 14. Accordingly, the need for adhesives or sealants between the septum 18 and body portion 12 may be avoided. However, the use of adhesives or sealants is not outside the contemplation of the present disclosure.

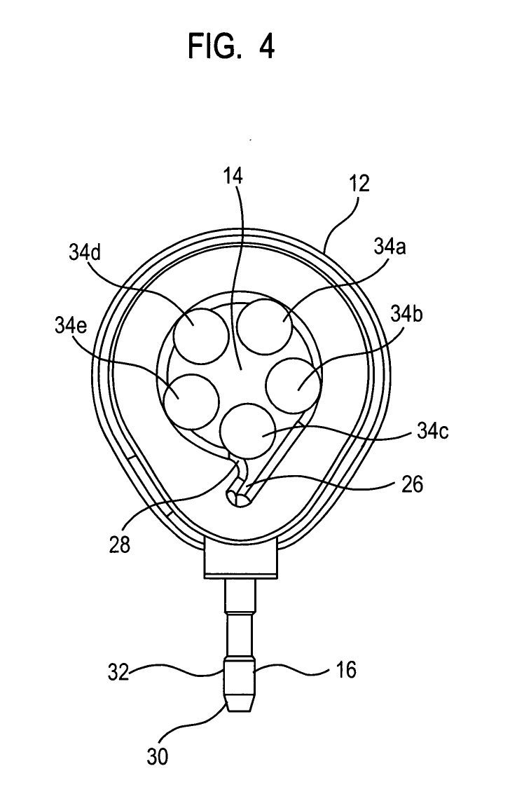

In plan view, the reservoir 14 of the exemplary access portal 10 may be provided having a generally circular shape, as shown in FIG. 4. The illustrated circular shape is merely exemplary, however. The reservoir 14 may be provided having various other shapes, such as an oval. The reservoir 14 may generally be defined by a sidewall 20 and a bottom 22. As shown the side wall 20 and the bottom 22 may meet in a radiused junction 24. The radiused junction 24 may reduce hang-up or stagnation of fluid passing into or out of the reservoir 14.

The reservoir 14 may include an outlet passage 26 extending from the reservoir 14 and in communication with the stem 16. The outlet passage 26 may extend from the reservoir 14 at an angle relative to the sidewall 20. As best seen in FIG. 4, in the illustrated exemplary embodiment the outlet passage 26 may extend from the reservoir 14 to provide a generally tangential outlet passage. The angled outlet passage 26 may reduce turbulence of fluids entering or exiting the reservoir 14 through the passage 26. Also as shown, a rounded transition 28 may be provided between the passage 26 and the reservoir 14. The rounded transition 28 between the reservoir 14 and the passage 26 may facilitate smooth flow between the reservoir 14 and passage 26. The combination of the angled passage 26 and the rounded transition 28 may also reduce the occurrence of hang-up and/or dead spots, i.e., regions that are not ready cleared by the flow of fluid through the access portal, adjacent the passage 26. While the passage 26 of the illustrated embodiment is shown as a generally linear extension, it should be understood that the passage 26 may be an arcuate extension.

The radiused junction 24 between the sidewall 20 and bottom 22 of the reservoir provides smooth transitions for fluid moving in the reservoir 14. The smooth transition may reduce the occurrence of turbulent flow of fluid entering or exiting the reservoir. The radiused junction 24 may also reduce hang-up of fluid, i.e., localized stagnation of fluid. Additionally, the angled orientation of the outlet passage 26 and rounded junction 28 between the passage 26 and the reservoir 14 may also reduce turbulent flow of fluid entering or exiting the reservoir 14. Reducing turbulent flow of fluid entering or exiting the reservoir 14, and reducing hang-ups and/or regions of stagnation within the access portal 10 may produce a number of effects.

Various fluids that may be infused or aspirated using an access portal may produce a precipitate or a residue if they are allowed to stagnate. As one example, any blood that becomes hung-up in the access portal 10, or is otherwise allowed to stagnate may coagulate inside the reservoir 14. The radiused junction 24 between the bottom 22 and sidewall 20 of the reservoir 14, as well as the angled arrangement of the outlet passage 26 and the rounded transition 28 into the passage 26 may generally reduce turbulent flow of fluids entering or exiting the reservoir. Additionally, at least some of these aspects of the exemplary port may also reduce the hang-up of fluid or stagnant regions within the reservoir 14. Reducing turbulence of fluids entering and/or exiting the reservoir 14 and reducing hang-up or stagnant regions may facilitate efficient clearing of fluids from the reservoir 14 and flushing of the reservoir 14. Accordingly, the accumulation of precipitates in the access portal 10 may also be reduced.

Flushing an access portal 10 may include introducing a flushing fluid into the reservoir 14 of the access portal 10. The flushing fluid introduced into the reservoir 14 may displace any fluid, and/or any solid matter, e.g., precipitate, initially in the reservoir out of the access portal 10. The displaced fluid may pass from the access portal 10 into the vascular system of the patient. During flushing, the flushing fluid may also mix with the fluid initially in the reservoir 14. Because of the mixing between the flushing fluid and the fluid initially in the reservoir 14, it may be necessary to flush the portal 10 with a quantity of flushing fluid equal up to several times the volume of the portal 10.

According to another aspect, the disclosure is directed at an access port 10 that provides improved flushing efficiency by reducing the fluid fill volume, e.g., free volume. As used in any embodiment herein, the fluid fill volume of the access portal is the volume of fluid that may be contained in the portal. Consistent with the exemplary embodiment, the fluid fill volume of the reservoir 14 may be reduced without reducing the depth of needle penetration or the area of the reservoir 14 that may receive a needle.

Turning to FIG. 4, the illustrated exemplary portal 10 includes five spherical volume reduction members 34a-34e disposed in the reservoir 14. The volume reduction members 34a-34e act to reduce the fluid fill volume of the reservoir 14. The volume reduction members 34a-34e may be retained in the reservoir 14 by the septum (not shown in FIG. 4). Additionally, as shown the volume reduction members 34a-34e may be sized and/or shaped to prevent the volume reduction members 34a-34e from obstructing the outlet passage 26. The volume reduction members 34a-34e may be formed from any suitable material, including glass, plastic, stainless steel, titanium, ceramic, etc. According to one embodiment, the volume reduction members 34a-34e may be formed from a hard material that is not prone to producing debris when subjected to needle strikes.

In the illustrated embodiment the volume reduction members 34a-34e are disclosed having a spherical shape. It should be understood, however, that the spherical shape is not essential. The volume reduction members 34a-e may be provided in various other shapes. For example, the volume reduction members 34a-e may be prismatic bodies, cylinders, egg shaped, etc. Similarly, while the exemplary access portal 10 includes five volume reduction members 34a-34e any number of members may be used to provide a reduction in the fluid fill volume of the access portal.

According to one embodiment the volume reduction members 34a-34e may be movable within the reservoir 14. Consistent with this embodiment, the volume reduction members 34a-34e may be displaced under an applied load. For example, when the portal 10 is accessed by a needle inserted through the septum 18, the needle may strike one of the volume reduction members 34a-34e. If the volume reduction members 34a-34e are movable within the reservoir the volume reduction members 34a-34e may be displaced by the force of the needle. Accordingly, the depth of penetration of a needle accessing the portal is not restricted in the exemplary embodiment.

According to another embodiment, the volume reduction members 34a-34e may be non-movable within the reservoir 14. According to such an embodiment, if a needle being inserted to access the portal 10 strikes one of the volume reduction members 34a-34e the needle may deflect around the object. For example, in the case of the exemplary spherical volume reduction members 34a-34e, a needle striking one of the volume reduction members 34a-34e the tip of the needle may slide along the arcuate surface of the volume reduction members 34a-34e, causing the needle to deflect around the volume reduction member 34a-e. Deflecting around a volume reduction member 34a-34e within the reservoir 14 may involve changing the angle of attack of the needle, whereby the needle may extend into the reservoir into a space between the volume reduction members 34a-34e. Needle insertion may be facilitated by providing the non-movable volume reduction members 34a-e having an angled or arcuate upper surface. For example, the volume reduction members 34a-e may be provided as pyramids, cones, spheres, hemi-spheres, etc.

In a portal 10 having non-movable volume reduction members 34a-e, the volume reduction members 34a-e may be integrally formed with the body portion 12. For example, the volume reduction members 34a-e may be formed as projections from the bottom 22 of the reservoir 14, from the sidewall 20 of the reservoir 14, or a combination of both. According to another embodiment, the non-movable volume reduction members 34a-e may be separate components that are positioned in a non-movable condition within the reservoir.

Reducing the fluid fill volume of the reservoir consistent with this preceding disclosure may allow the portal reservoir to be cleared or flushed with less fluid. For example, a reservoir having a total volume of 1 ml achieves a ten times exchange from a flushing volume of 10 ml of saline. If the fluid fill volume of the reservoir is reduced to 0.5 ml, flushing the port with 10 ml of saline will achieve a twenty times exchange. Accordingly, by reducing the fluid fill volume of the reservoir, the port may be more thoroughly flushed without introducing a greater amount of fluid into the patient. Additionally, if the access portal is used for aspiration, a smaller fluid fill volume of the reservoir may allow less fluid to be drawn into the reservoir.

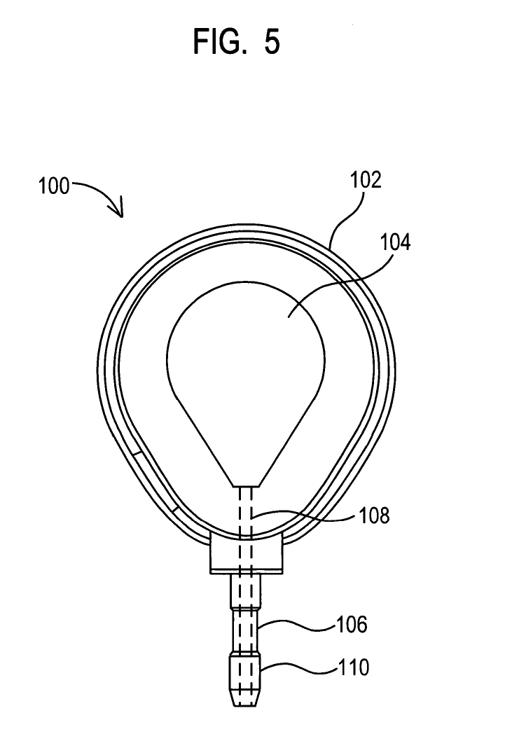

Turning next to FIG. 5, another embodiment of an access portal 100 providing efficient flushing is shown. The illustrated access portal 100 may generally include a body 102 defining a fluid reservoir 104 therein. The access portal 100 may also include a stem 106 in fluid communication with the fluid reservoir 104. As with the preceding embodiments, the access portal consistent with the embodiment illustrated in FIG. 5 may additionally include a housing member.

As with previously described embodiments, the various components of the access portal 100 may be produced from a variety of biocompatible materials including metallic materials, ceramic materials, polymeric materials, and combinations thereof. For example, the body 102 of the access portal 100 may be formed from a polymeric material and may include a metallic cup, liner, or bottom plate making up at least a portion of the reservoir 104, thereby reducing the production of debris resulting from needle strikes in the reservoir 104. Consistent with other embodiments, the entire body 102 of the access portal 100 may be formed from a polymeric material, either as a unitary construction or as an assembly of components. Various other materials and constructions may also suitably be employed for producing an access portal 100 consistent with the present disclosure.

As with previous embodiments, the fluid reservoir 104 of the access portal 100 may be configured having an open top to provide access to the reservoir 104. While not shown in the plan view illustration, the access portal 100 may additionally include a septum disposed over the fluid reservoir 104. The septum may restrict fluid passage to and from the reservoir 104 through the open top thereof, but may allow the reservoir 104 to be accessed, e.g., by a needle penetrating the septum.

The stem 106 may provide fluid communication between the fluid reservoir 104 and the exterior of the access portal 100, thereby allowing fluids to be delivered from the fluid reservoir 104 to a predetermined location in the body, or to be extracted from a predetermined location in the body through the fluid reservoir 104. A fluid passageway 108 may be provided extending from the fluid reservoir 104 and through the stem 106, thereby providing fluid communication from the fluid reservoir 104. As with previous embodiments, the stem 106 may be provided with a bulbous end portion 110 to facilitate securing a catheter (not shown) to the stem 106. A catheter secured to the stem 106 may implanted in the body of a patient extending from the access portal 100 to a predetermined location in the body of the patient. Accordingly, the catheter may allow fluid to be delivered to, or extracted from, such predetermined location in the body of a patient.

Consistent with the illustrated embodiment, the fluid reservoir 104 may be provided having a teardrop shape in plan view. That is, with reference to FIG. 5, the fluid reservoir 104 may be shaped having a generally arcuate contour that converges towards a single point on one side. Consistent with the illustrated embodiment, the fluid reservoir 104 may have an arcuate shape that converges toward a single point in the region of the fluid passageway 108 of the stem 106. Accordingly, fluid introduced into the fluid reservoir 104, e.g. through the septum by a needle, may be directed toward the fluid passageway 108 by the contour of the fluid reservoir 104. The continuous contour of the fluid reservoir 104 may have few, or no, hard angles or inside corners and may, therefore, minimize turbulent flow of a fluid passing between the fluid reservoir 104 and the fluid passageway 108. Additionally, the lack of hard angles may minimize the occurrence of hang-up. That is, fluid may pass between the fluid reservoir 104 and the fluid passageway 108 without becoming, entrapped in a region of turbulent flow or a region outside of the flow path between the point of introduction and the fluid passageway 108.

Consistent with a further aspect, the junction between the bottom of the fluid reservoir 104 and the sidewalls of the reservoir 104 may be rounded, thereby reducing any flow drag, turbulence, or hang-ups. Similarly, the side of the septum facing the interior of the fluid reservoir 104 may include a contoured or rounded transition between the septum and the sidewalls of the fluid reservoir 104. The contour or rounded transition may also eliminate hard corner between the septum and the sidewall of the reservoir 104. According to one embodiment, the fluid reservoir 104 may be tapered across the depth thereof and/or include a tapered region adjacent the fluid passageway 108 in communication with the reservoir 104. The tapering of the fluid reservoir 104 may generally provide a smooth transition between the reservoir 104 and the fluid passageway 108. Accordingly, the geometry of the fluid reservoir 104 and the transition between the fluid reservoir 104 and the fluid passageway 108 may be configured to minimize or eliminate any features that may cause hang-up and/or may be optimized to reduce or eliminate any causes of turbulent flow between the fluid reservoir 104 and the fluid passageway 108.

In addition to including a reservoir 104 having a geometry that may reduce or eliminate hang-up and turbulent flow, the access portal 100 may also include one or more volume reducing members disposed within the reservoir 104. Accordingly, in addition to eliminating impediments to smooth flow into and out of the reservoir 104, the access portal may also provide a reduced internal volume. The one or more volume reducing members may be configured and arranged in a manner as described with reference to the preceding embodiments.

There is thus provided a vascular access portal that may provide more efficient flushing and may reduce the accumulation of precipitates and/or residue in the access portal. An access portal consistent with the present disclosure may generally include a housing, and a body defining a fluid reservoir. A septum may be disposed on the housing, thereby enclosing the fluid reservoir. A stem may be provided in fluid communication with the fluid reservoir through an outlet passage extending form the fluid reservoir. The access portal may also include at least one volume reduction member disposed within the fluid reservoir. The volume reduction member within the fluid reservoir may reduce the fluid fill volume of the fluid reservoir. According to one embodiment, the outlet passage extending providing fluid communication between the fluid reservoir and the stem may extend from the fluid reservoir at an angle. Furthermore, the outlet passage may extend tangentially from the fluid reservoir. In a first exemplary embodiment, the object disposed within the fluid reservoir may be movable within the reservoir. In a second exemplary embodiment, the object within the fluid reservoir may be fixed. According to yet another embodiment, the fluid reservoir may have a teardrop shape that promotes fluid passage between the stem and the reservoir while minimizing hang-up within the reservoir.

The description hereinabove is directed at exemplary embodiments consistent with the invention. It should be understood, however, that the described embodiments are susceptible to modification and variation without materially departing from the invention set forth in the claims appended hereto.

What is claimed is:

1. An access portal comprising:
a housing,
a body defining a fluid reservoir;
a septum enclosing said fluid reservoir;
a stem in fluid communication with said fluid reservoir via an outlet passage; and
at least one member disposed in said fluid reservoir, said member reducing a fluid fill volume of said reservoir.

2. An access portal according to claim 1, wherein said outlet passage extends' from said fluid reservoir at an angle.

3. An access portal according to claim 2, wherein said outlet passage extends tangentially from said fluid reservoir.

4. An access portal according to claim 1, wherein said at least one object is movably disposed in said fluid reservoir.

5. An access portal according to claim 1, wherein said at least one object comprises a sphere.

6. An access portal according to claim 1, wherein said at least one member is non-movable within said fluid reservoir.

7. An access portal according to claim 6, wherein said at least one member is integrally formed with said fluid reservoir.

8. An access portal comprising:
a reservoir containing at least one spherical member movably disposed within said reservoir;
a septum enclosing said reservoir; and
a stem providing fluid communication with said reservoir.

9. An access portal according to claim 8, further comprising a housing, said septum being disposed between said housing and said reservoir.

10. An access portal comprising
a housing,
a body defining a fluid reservoir;
a septum enclosing said fluid reservoir; and
a stem in fluid communication with said fluid reservoir via an outlet passage;
said fluid reservoir having an arcuate shape converging to a point at said outlet passage.

11. An access portal according to claim 10 wherein a depth of said fluid reservoir tapers toward said outlet passage.

12. An access portal according to claim 10, said fluid reservoir comprising a bottom and at least one sidewall, wherein said bottom and sidewall comprise a rounded junction.

13. An access portal according to claim 10 comprising a rounded transition between said septum and said fluid reservoir.

14. An access portal according to claim 10 comprising at least one member disposed in said fluid reservoir, said member reducing a fluid fill volume of said fluid reservoir.

15. An access portal according to claim 14 wherein said at least one object is movably disposed in said fluid reservoir.

16. An access portal according to claim 14 wherein said at least one object comprises a sphere.

17. An access portal according to claim 14 wherein said at least one member is integrally formed with said fluid reservoir.

18. An access portal comprising:
a housing;
a body defining a fluid reservoir;
a septum enclosing said fluid reservoir; and
a stem in fluid communication with said fluid reservoir via an outlet passage, said outlet passage extending tangentially from said fluid reservoir.

19. An access portal according to claim 18 comprising at least one member disposed in said fluid reservoir, said member reducing a fluid fill volume of said reservoir.
